(19) United States
(12) Patent Application Publication
(10) Pub. No.: US 2026/0124468 A1
(43) Pub. Date: May 7, 2026

(54) RADIOTHERAPY SYSTEMS WITH POSITION CONTROL AND METHODS OF CONTROLLING RADIOTHERAPY SYSTEMS

(71) Applicant: Varian Medical Systems, Inc., Palo Alto, CA (US)

(72) Inventors: Joerg FREUDENBERGER, Kalchreuth (DE); Anja FRITZLER, Erlangen (DE)

(73) Assignee: Varian Medical Systems, Inc., Palo Alto, CA (US)

(21) Appl. No.: 18/938,389

(22) Filed: Nov. 6, 2024

(51) Int. Cl. A61N 5/10; G16H 20/40; G16H 40/63

(52) U.S. Cl. CPC A61N 5/1067; A61N 5/1045; A61N 5/1049; A61N 5/1081; G16H 20/40; G16H 40/63; A61N 2005/1089

(57) ABSTRACT

One or more example embodiments provides a radiotherapy system including a radiation system configured to generate a plurality of radiation beams and emit radiation to a patient, the emitted radiation including the plurality of radiation beams; a detector configured to detect at least a portion of emitted radiation that passes through the patient; and a controller configured to control movement of at least a portion of the radiation system based on the detected radiation.

### TECHNICAL FIELD

Example embodiments relate to radiotherapy systems with position control and methods of controlling the radiotherapy systems.

### BACKGROUND

The use of radiation therapy to treat cancer is well known. Typically, radiation therapy involves directing a beam of high energy proton, photon, ion, or electron radiation (“therapeutic radiation”) into a target or target volume (e.g., a tumor or lesion).

FLASH radiation therapy delivers an entire, relatively high therapeutic radiation dose to a target within a single, short period of time.

Microbeam radiation therapy (MBT) delivers a plurality of radiation fields (microbeams) that are spaced apart from each other.

### SUMMARY

One or more example embodiments relate to a radiotherapy system including a radiation system configured to generate a plurality of radiation beams and emit radiation to a patient, the emitted radiation including the plurality of radiation beams; a detector configured to detect at least a portion of emitted radiation that passes through the patient; and a controller configured to detect movement of the patient based on the detected radiation and control movement of at least a portion of the radiation system based on the detected movement of the patient.

According to one or more example embodiments, the radiation system is configured to generate the plurality of radiation beams to be parallel and emit the plurality of radiation beams in parallel to the patient.

According to one or more example embodiments, the controller is configured to control the radiation system such that the plurality of radiation beams deliver a radiation rate of less than 40 grays per second (Gy/s).

According to one or more example embodiments, the controller is configured to control the radiation system such that the plurality of radiation beams emit the radiation to the patient for greater than 2 seconds.

According to one or more example embodiments, the controller is configured to reconstruct a first image based on detected radiation at a first instance and reconstruct a second image based on detected radiation at a second instance, determine a difference between the first image and the second image, and control the movement of the portion of the radiation system based on the determined difference.

According to one or more example embodiments, the detected radiation is separate from the plurality of radiation beams.

According to one or more example embodiments, the radiation system includes a radiation source configured to emit a first radiation beam onto a target to form a focal spot on the target, the target configured to convert the first radiation beam into a radiation field, the focal spot being a source of the radiation field; and a collimator configured to convert the radiation field into at least the plurality of radiation beams.

According to one or more example embodiments, the radiation system further includes a deflector configured to alter the first radiation beam, wherein the controller is configured to control the deflector based on the detected radiation to change a location of the focal spot.

According to one or more example embodiments, the controller is configured to control the radiation system such that the plurality of radiation beams deliver a reduced radiation rate when the location of the focal spot is changing.

According to one or more example embodiments, the target is a cylinder having a diameter less than 50 cm.

One or more example embodiments relates to a method of controlling movement of at least a portion of a radiotherapy system, the method including generating a plurality of radiation beams and emitting radiation to a patient, the emitted radiation including the plurality of radiation beams; detecting at least a portion of emitted radiation that passes through the patient; and controlling movement of the portion of the radiotherapy system based on a detected movement of the patient, the controlling the movement of the portion of the radiotherapy including detecting the movement of the patient based on the detected radiation.

According to one or more example embodiments, the generating generates the plurality of radiation beams to be parallel and the emitting emits the plurality of radiation beams in parallel to the patient.

According to one or more example embodiments, the controlling controls the radiotherapy system such that the plurality of radiation beams deliver a radiation rate of less than 40 grays per second (Gy/s).

According to one or more example embodiments, the controlling controls the radiotherapy system such that the plurality of radiation beams emit the radiation to the patient for greater than 2 seconds.

According to one or more example embodiments, the controlling includes reconstructing a first image based on detected radiation at a first instance and reconstructing a second image based on detected radiation at a second instance, determining a difference between the first image and the second image, and controlling the movement of the portion of the radiotherapy system based on the determined difference.

According to one or more example embodiments, the detected radiation is separate from the plurality of radiation beams.

According to one or more example embodiments, the generating includes emitting a first radiation beam onto a target to form a focal spot on the target, converting the first radiation beam into a radiation field, the focal spot being a source of the radiation field, and converting the radiation field into at least the plurality of radiation beams.

According to one or more example embodiments, the controlling controls the radiotherapy system such that the plurality of radiation beams deliver a reduced radiation rate during the altering.

According to one or more example embodiments, the controlling includes altering the first radiation beam using a deflector, and controlling the deflector based on the detected radiation to change a location of the focal spot.

### BRIEF DESCRIPTION OF THE DRAWINGS

Other objects and features of the present invention will become apparent from the following detailed description considered in conjunction with the accompanying drawings.

The drawings, however, are only examples and schematic solely for the purpose of illustration and do not limit example embodiments. In the drawings:

FIG. 1 illustrates a radiotherapy system according to one or more example embodiments;

FIGS. 2A-2B illustrate a radiation system and an example beam path for microbeam radiation therapy (MBT) according to one or more example embodiments;

FIG. 3A illustrates a position control system within the radiotherapy system of FIG. 1, according to one or more example embodiments;

FIG. 3B illustrates a diagram for changing a deflection of a focal spot using the position control system of FIG. 3A according to one or more example embodiments;

FIG. 4 illustrates a method of controlling at least a portion of the radiotherapy system according to one or more example embodiments;

FIG. 5 illustrates a method of determining a difference between images according to one or more example embodiments; and

FIG. 6 illustrates a block diagram of a control system with which embodiments may be implemented.

### DETAILED DESCRIPTION

Various example embodiments will now be described more fully with reference to the accompanying drawings in which only some example embodiments are shown. Specific structural and functional details disclosed herein are merely representative for purposes of describing example embodiments. Example embodiments, however, may be embodied in various different forms, and should not be construed as being limited to only the illustrated embodiments. Rather, the illustrated embodiments are provided as examples so that this disclosure will be thorough and complete, and will fully convey the concepts of this disclosure to those skilled in the art. Accordingly, known processes, elements, and techniques, may not be described with respect to some example embodiments. Unless otherwise noted, like reference characters denote like elements throughout the attached drawings and written description, and thus descriptions will not be repeated. The present invention, however, may be embodied in many alternate forms and should not be construed as limited to only the example embodiments set forth herein.

Detailed illustrative embodiments are disclosed herein. However, specific structural and functional details disclosed herein are merely representative for purposes of describing example embodiments. The example embodiments may, however, be embodied in many alternate forms and should not be construed as limited to only the embodiments set forth herein.

When the words “about” and “substantially” are used in this application in connection with a numerical value, it is intended that the associated numerical value include a tolerance of +10% around the stated numerical value, unless otherwise explicitly defined. Further, regardless of whether numerical values are modified as “about” or “substantially,” it will be understood that these values should be construed as including a of +10% around the stated numerical value.

Independent of the grammatical term usage, individuals with male, female or other gender identities are included within the term.

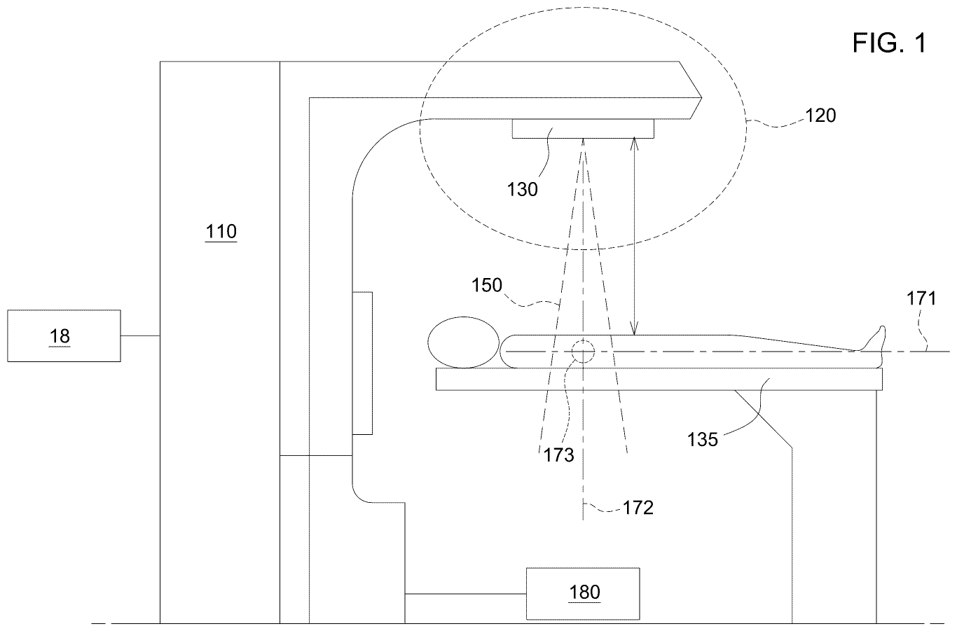

FIG. 1 illustrates a block diagram of an exemplary radiotherapy system 100 in accordance with one or more example embodiments. Radiotherapy system 100 may be similar to a Varian TrueBeam® radiotherapy system, commercially available from Varian Medical Systems, Palo Alto, Calif.

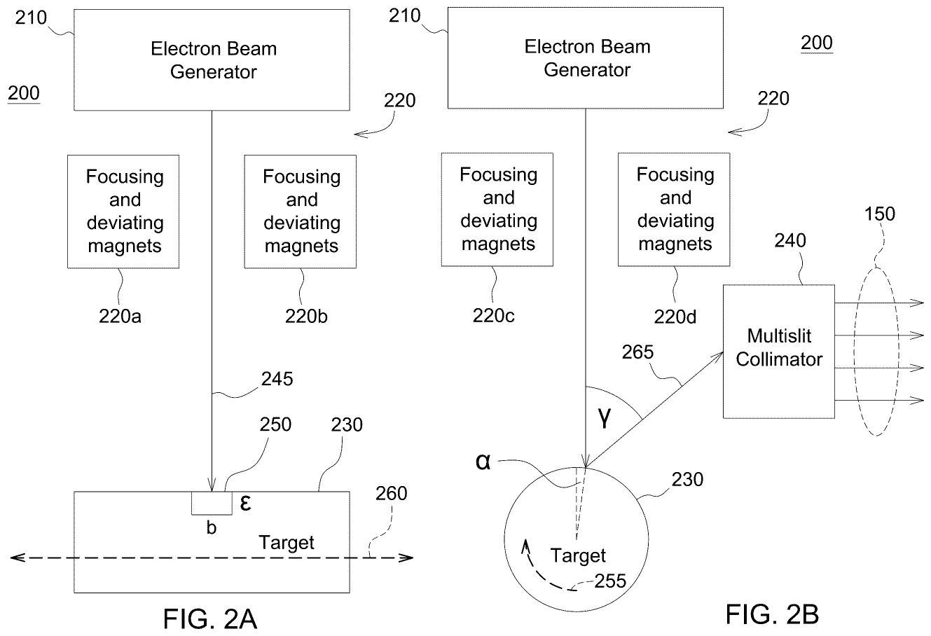

Stand 110 supports a rotatable gantry 120 with a treatment head 130. The treatment head 130 may extend into the gantry 120. In proximity to stand 110 there is arranged a controller 18 which includes control circuitry for controlling the different modes of operation of the radiotherapy system 100. In one embodiment, treatment head 130 includes a multislit collimator (MSC) (shown in FIG. 2).

Radiotherapy system 100 comprises a radiation system, for example, within gantry 120, utilized to create a radiation beam. Typically, radiotherapy system 100 is capable of generating either an electron (particle) beam or an X-ray (photon) beam for use in the radiotherapy treatment of patients on a treatment couch 135. A high voltage source is provided within the stand 110 and/or in the gantry 120 to supply voltage to an electron gun (not shown) positioned on an accelerator guide located in the gantry 120. Electrons are emitted from the electron gun into an accelerator where they are accelerated. A source supplies radio frequency (microwave) power for the generation of an electric field within the waveguide. The electrons emitted from the electron gun are accelerated in the waveguide by the electric field and exit the waveguide as a high-energy electron beam for example, at megavoltage energies. The electrons impact a bremsstrahlung target and photon radiation is produced. In one embodiment, the gantry includes a component (e.g., bend magnets, etc.) for redirecting the beams (e.g., in the direction of a patient, etc.).

As illustrated in FIG. 1, a patient is shown lying on the treatment couch 135. The multiple radiation beams 150 are emitted from the treatment head 130 (e.g., as described above, etc.) towards the patient. In some example embodiments, the beams 150 may be microbeams (e.g., radiation fans), each having a thickness of 50-75 μm and separated from each other by at least 100 μm. However, example embodiments are not limited thereto. For example, the microbeams may have a fan geometry with a width (e.g., a thickness) of 30-200 μm at the isocenter 173 and separated from each other by at least twice the width, for example, 100 μm. The width may be considered parallel to the patient plane 171. In other example embodiments, other geometric beams may be used.

In an x-ray implementation, a patient plane 171 is usually positioned about one meter from the X-ray target (shown in FIG. 2), and the rotational axis of the gantry 120 is located on the patient plane 171, such that the distance between the target and the isocenter 173 remains constant when the gantry 120 is rotated.

The isocenter 173 is at the intersection between the patient plane 171 and the central axis 172 of radiation beams 150. A treatment volume to be irradiated may be located about the isocenter 173. It is appreciated that some treatment plans may utilize a primary target that is off of the central beam axis, and such arrangements are within the scope of embodiments in accordance with some example embodiments.

The radiotherapy system 100 further includes a detector 180, such as an imager, located at an operative position relative to the treatment head 130 (e.g., under the treatment couch 135).

Current research in radiation oncology deals with microbeam radiation therapy (MBT) and FLASH, among others. MBT uses radiation fields including radiation fans with a thickness of 50-75 μm. These radiation fans have a distance of several 100 μm from each other. Studies show that tissue in the radiation compartments tolerates high doses and tumor growth (cancer) can be well controlled in animal experiments. In order to exclude camera shake, the aim is to use very high dose rates, so that the desired dose is typically achieved in less than a second in the tissues irradiated in the radiation compartments. This results in extreme demands on the radiation source, which are currently only achieved with synchrotron systems. FLASH describes irradiation applications in which the dose rate is greater than 40 Gy/s. In animal experiments, it has been observed that tumors can also be better controlled in their growth with this radiation.

In the MBT methods envisaged today, dose rates are in the FLASH range (>40 Gy/s).

At least some example embodiments are directed to achieving the spatially very high fractionation of MBT with lower dose rates (e.g., without FLASH) and, thus, enable the use of technically simpler radiation sources. By monitoring the position of the radiation fields with MBT, the location of the radiation fields can be controlled and delivered for a longer time while still avoiding non-targeted areas of the patient.

FIGS. 2A-2B illustrate a radiation system and an example beam path for microbeam radiation therapy (MBT) according to one or more example embodiments.

As shown in FIGS. 2A-2B, a radiation system 200 includes an electron beam generator (a radiation source) 210, a deflector 220 (shown as focus and deviating magnets 220a-220d in FIGS. 2A-2B), a target 230 and a multislit collimator (MSC) 240. The radiation system 200 may be included in the radiotherapy system 100 and may be controlled by the controller 18. The radiation system 200 may include additional elements than those shown in FIGS. 2A-2B. The radiation system may be the system described in Bartzsch, et al., Line focus x-ray tubes—a new concept to produce high brilliance x-rays, Phys. Med. Biol. 62, pp. 8600-8615 (2017), the entire contents of which are incorporated by reference.

The electron beam generator 210 generates and accelerates a first radiation beam toward the target 230. In this example, the first radiation beam 245 is an electron beam. However, example embodiments are not limited to electron beam generation.

In further embodiments, the electron beam generator 210 may be a treatment radiation source for providing treatment energy, wherein the treatment energy may also be used to obtain images. In such cases, in order to obtain imaging using treatment energies, the detector 180 is configured to generate image data in response to radiation having treatment energies. In some embodiments, the treatment energy is generally those energies of 160 kilo-electron-volts (keV) or greater and diagnostic energy is generally those energies below the high energy range, and more typically below 160 keV. In other embodiments, the treatment energy and the diagnostic energy can have other energy levels and refer to energies that are used for treatment and diagnostic purposes, respectively. In some embodiments, the radiation system 200 is able to provide X-ray radiation at a plurality of photon energy levels within a range anywhere between approximately 10 keV and approximately 20 MeV.

The focus and deviating magnets 220a-220d electromagnetically shape the first radiation beam 245 to form a focal spot 250 on the target 230. The focus and deviating magnets 220a-220d are controlled by the controller such that the first radiation beam 245 forms a desired focal spot, in size (e.g., a desired height b and a desired width &) and location. In magnetic deflection, there is a coil carrying a current in the vicinity of the radiation beam generating a magnetic field at the volume where the electron beam travels through. By changing the magnetic field strength, the electron beam is deflected by the “Lorenz force.”

While magnetic deflection is described in the example of an X-ray tube and controlling movement of an electron beam, example embodiments are not limited thereto. For example, a source location of a laser beam may be controlled in the case of a Compton backscattering source. For Compton backscattering, the focal spot is defined as the cross-section of the interaction of electrons with a laser. The deflector is able to shift both the electron beam and the laser. Therefore, the target would be an interaction volume of electrons and photons.

In some example embodiments, the target 230 is a cylinder having a diameter of less than 50 cm. In some example embodiments, the target 230 may have a radius of 10 cm and a length of 30 cm. The controller 18 controls the target 230 to rotate in a direction 255 about a rotational axis 260. The rotational axis 260 may extend in a longitudinal direction of the target 230. In addition, the controller 18 controls the target 230 to move the target 230 linearly in both directions along the rotational axis 260 in a reciprocating manner.

The target 230 may be an anode target and made of tungsten or any other suitable material. In an X-ray tube, X-ray radiation is produced by the incidence of an electron beam on an anode. The incident electrons define a focal spot. In the case of a rotating anode, a focal path is produced during operation of the X-ray tube.

The target 230 converts the first radiation beam 245 into a photon radiation field 265 (e.g., an x-ray radiation field). More specifically, electrons are accelerated (e.g., up to a kinetic energy of 600 keV), hit the target in the focal spot (e.g., 30 mm×100 μm) at an incidence angle α (e.g.,) 10° and converted into photons to form the photon radiation field 265. The photon radiation field 265 is emitted from the target 230 at an emission angle γ (e.g.,) 60°.

While FIG. 1 is described relative to an X-ray generator operating around 500-600 keV, it should be understood that example embodiments are not limited thereto. For example, other energy levels generated by linear accelerators (LINACs) may be used. In other example embodiments, line focus tubes, microfocus tubes, Compton backscattering sources may be used.

Moreover, in some example embodiments a pixelated emitter may be used and the electron beam may be shifted/moved by moving a location of the electron emission at the electron emitter instead of magnetic deflection. Pixelated emitters can switch on to off status. To move the electron beam by a certain distance a pixel associated with the current beam may be turned off and a pixel associated with the new location may be turned on.

The MSC 240 converts the radiation field 265 into the plurality of radiation beams 150. The MSC 240 may have multiple slits (e.g., each 50 μm wide and 30 mm high slit apertures with a pitch of 400 μm) to the generate the plurality of radiation beams 150 (e.g., microbeams, which may also be referred to as radiation fans).

The MSC 240 may have an area including a pre-filter to attenuate radiation. In this example, a collimator with a high shaft ratio can be closer to the target as well as the treatment volume, resulting in an improved usage of the dose and reducing possible expansion of individual beams. A shaft ratio may be a ratio of a height of the collimator (usually equal to the thickness in the direction of the radiation) divided by a width of the open part of the collimator (which is the width of the microbeam at the exit of the collimator).

In other example embodiments, the MSC 240 may include an additional filter on a side facing the patient.

The plurality of radiation beams 150 may be emitted to a patient and the controller 18 is configured to control the radiation system 200 such that the plurality of radiation beams 150 deliver a radiation rate of less than 40 grays per second (Gy/s) and such that the plurality of radiation beams 150 emit radiation to the patient for greater than 2 seconds.

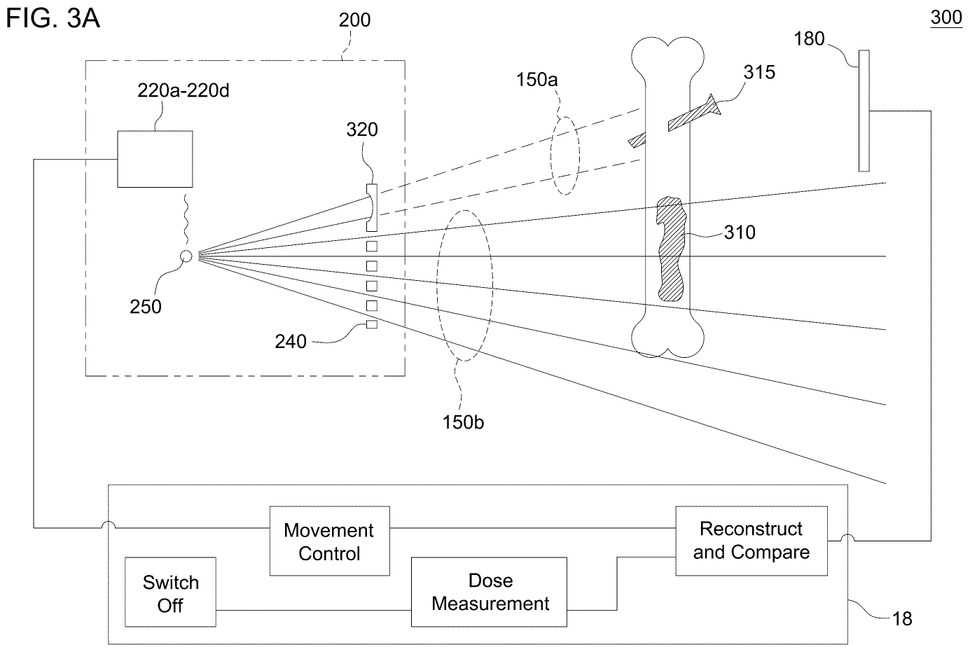

FIG. 3A illustrates a position control system within the radiotherapy system of FIG. 1, according to one or more example embodiments.

As shown in FIG. 3A, a position control system 300 includes the controller 18, the radiation system 200 and the detector 180.

As described above with respect to FIGS. 1-2, the radiation system 200 generates the plurality of radiation beams 150 and emits the beams to a treatment volume 310 (e.g., a tumor) of the patient. The plurality of beams 150 includes measurement beams 150a and therapeutic radiation beams 150b. The measurement beams 150a are a result of passing the photon radiation field 265 passing through an attenuator 320 instead of the collimator 240. The therapeutic beams 150b are a result of passing the photon radiation field 265 passing through the collimator 240. Thus, the measurement beams 150a have a lower dose rate compared to the therapeutic radiation and no microstructure is used in the measurement beams 150a.

Within a path of the measurement beams 150a is an artifact 315 within the patient. The artifact may be an item fixed within the patient and along the path of the measurement beams 150a. In some example embodiments, the artifact 315 is a screw. Additionally, or alternatively, the artifact 315 may be a bone edge, a screw. In other example embodiments, the artifact 315 may be secured to the patient (e.g., a metal plate taped to the patient) or table (e.g., when the patient is immobile or fixed to the table). However, example embodiments are not limited thereto. Artifacts may be an item having a high absorption contrast, i.e., the material of the artifact includes matter of high atomic number.

In at least some example embodiments, the controller 18 controls the radiation system 200 to generate the beams 150a and 150b. More specifically, the controller 18 controls the deflector 220 to move and shape the electron beam to change the location and/or size of the focal spot created by the electron beam.

During the emission of the beams 150a and 150b, the artifact 315 casts a shadow on the detector 180 as a result of some of the beams 150a being absorbed by the artifact and other beams avoiding the artifact 315, but striking the detector 180.

The detector 180 captures image data at various instances. For each instance, the image data is sent by the detector to the controller 18. The controller 18 reconstructs the image data into an image. While the reconstruction is shown as part of the controller 18, it should be understood that example embodiments are not limited thereto.

The controller 18 is configured to determine movement of the patient by determining a difference between a reconstructed image at a first instance to a reconstructed image at a second instance. More specifically, in some example embodiments, the controller 18 determines a difference in location of the artifact 315 between the first instance and the second instance. Based on the determined difference, the controller 18 controls the deflector to move and/or change the focal spot such that the positioning of the beams 150 relative to the patient returns to a target position relative to the patient during radiation treatment. For example, if the controller 18 determines there is a difference in the location of the artifact in the second instance from the first instance, the controller 18 controls the deflector (or pixelated emitter) such that the first radiation beam 245 forms the focal spot 250 on the target 230 in a position that results in the beams 150 having a same position relative to the patient (e.g., relative to the artifact 315) as the first instance (i.e., a target position relative to the patient or a starting position relative to the patient).

With a low dose, e.g., a topogram for orientation, the controller 18 can determine if the artifact 315 is in the target position relative to the beams 150. When the controller 18 detects movement of the artifact 315, the controller 18 may reduce the dose (radiation) until the controller 18 reports the artifact 315 is in the target position relative to the beams 150. With this procedure, it is possible to irradiate with an overall low dose rate and still achieve the (therapeutically necessary) dose in the irradiated volume of a few (5 and more) grays. The irradiation time can be significantly longer than one second.

By tracking a motion of the patient relative to the focal spot, microbeam radiotherapy can be used, e.g., using a low dose rate over an extended length of time.

In some example embodiments, the controller 18 controls the focal spot 250 to minimize and/or reduce the change in position of the shadow resulting from the artifact 315. If the position deviation is too large and/or a target dose is reached at the detector 180, the controller 18 can turn off the radiation system temporarily or for an extended time.

It is possible to include the movement of the organs in radiation planning. Movement control and the quick switching on and off could also enable motion control on mobile organs. When using a radiator equipped with a pixelated field effect emitter, the individual focal spots could be allowed to migrate directly and thus reduce the irradiation duration.

FIG. 3B illustrates a diagram for changing a deflection of a focal spot using the position control system of FIG. 3A according to one or more example embodiments.

As shown in FIG. 3B, the artifact 315 moves a displacement a (perpendicular to the central axis 172) which leads to a shift of its image at the detector 180 by a distance A.

The focal spot 250 and the collimator 240 are separated by a distance d and the focal spot 250 and the detector 180 are separated by a distance D. The distance d and the distance D extend in the general direction of the central axis 172 and are known system parameters.

The treatment area (e.g., tumor) and the focal spot 250 may be separated by a distance of αD, where a is less than 1. In other example embodiments, the distance αD may be between the patient and the focal spot 250.

A deflection Δf of the focal spot 250 while maintaining the position of the collimator leads to a shift of the radiation path as shown in FIG. 3B. More specifically, when the shift of the artifact 315 to position x is detected, the focal spot 250 is deflected by Δf resulting in beams 150_{Δf} that are shifted due to the deflection Δf of the focal spot 250.

The system 300 may control the deflection Δf of the focal spot 250 to keep the radiation pathways in the target volume as follows:

$$Δ f = \frac{α Ad}{α D - d}$$

where Δf is directed in the opposite direction with respect to the distance A.

In some example embodiments, the value a may initially be set to 0.5. In some example embodiments, the value a may be adjusted.

When the system 300 determines a motion of the patient (e.g., distance a), then the focal spot 250 is deflected by the control system 300 and, subsequently, a new position of the image of the artifact 315 on the detector 180 is stored.

In some example embodiments, when the system 300 determines that the deflection Δf (or a sum of all deflections) of the focal spot 250 exceeds a threshold, then system 300 may turn off the radiation.

For example, for the sum of all deflection steps F (i.e. the actual position of the beam compared to its position at the beginning of the treatment), the system 300 turns the radiation off if:
F>d*(width of a microbeam)/(Length of the radiation pathway through the tumor); or, in an example embodiment

$$F > d * ( width of a microbeam ) / ( 5 cm ) .$$

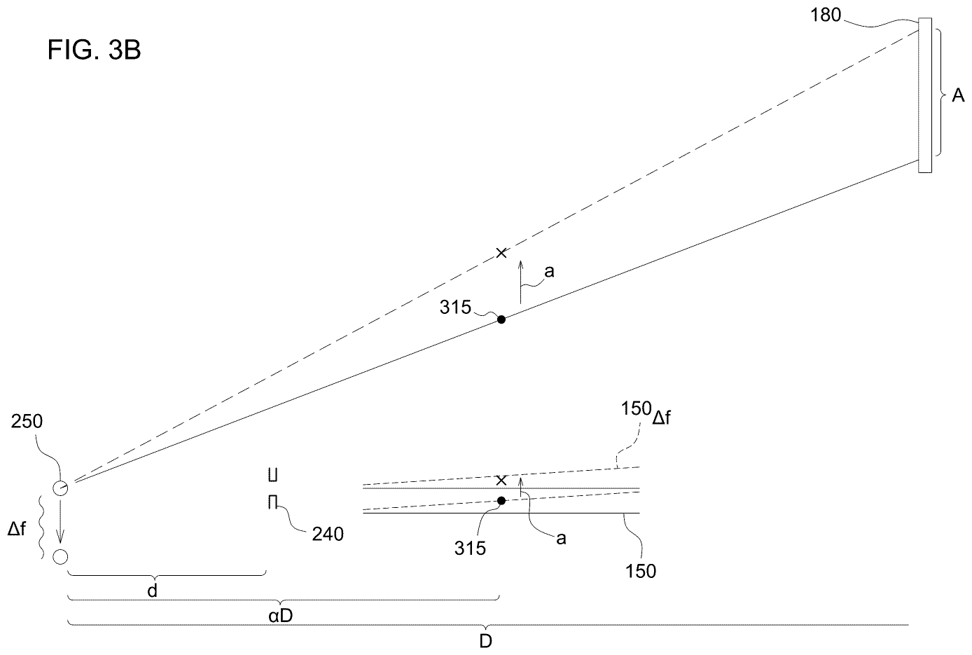

FIG. 4 illustrates a method of controlling at least a portion of the radiotherapy system according to one or more example embodiments. The method of FIG. 4 may be performed by the systems shown and described in FIGS. 1-3.

At S400, the controller causes the radiation system to emit radiation to the patient (e.g., the plurality of radiation beams 150).

At S405, the detector detects emitted radiation through the patient and provides the detected radiation to the controller.

At S410, the controller controls movement based on the detected movement of the patient, which is based on the detected radiation.

In at least some example embodiments, and as described above, the controller causes the system to move the beam 245 through magnetic deflection when movement is detected such that the initial position of the beams 150 relative to the patient may be resumed. In other embodiments, the controller causes the system to move the beam 245 using pixelated electron emitters.

In some example embodiments, the controller determines a difference between images to control movement of at least a portion of the radiation system.

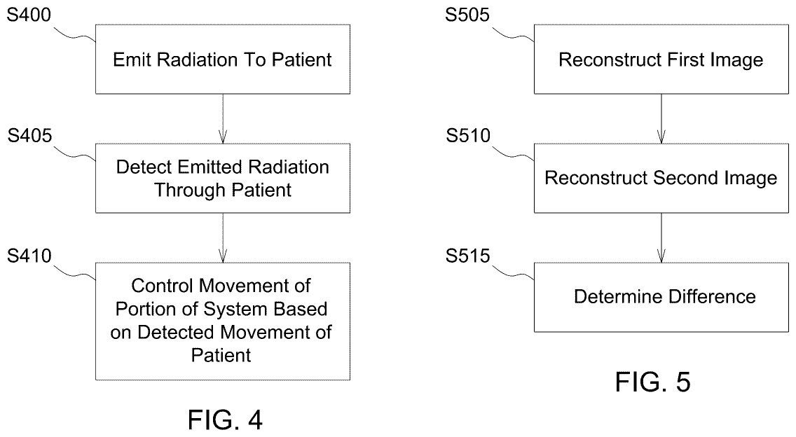

FIG. 5 illustrates a method of determining a difference between images according to one or more example embodiments. At S505, the controller reconstructs a first image using the detected radiation at a first time instance. At S510, the controller reconstructs a second image using the detected radiation at a second time instance. The controller may reconstruct the first and second images using any known image reconstruction technique.

At S515, the controller determines a difference between the first image and the second image. The controller may determine the difference using any known technique, for example, comparing a pixel value of the first image to a pixel value of the second image for a same location of the detector. The difference corresponds to a movement of the patient and/or system.

Referring back to FIG. 4, the controller controls the movement of at least a portion of the radiation system (e.g., the radiation beam 245) based on the determined difference at S410. For example, the controller moves a position of the focal spot (e.g., by controlling the focus and deviating magnets 220a-220d to change the incidence angle α and the emission angle γ) based on the determined difference.

None of the elements recited in the claims are intended to be a means-plus-function element within the meaning of 35 U.S.C. § 112 (f) unless an element is expressly recited using the phrase “means for” or, in the case of a method claim, using the phrases “operation for” or “step for.”

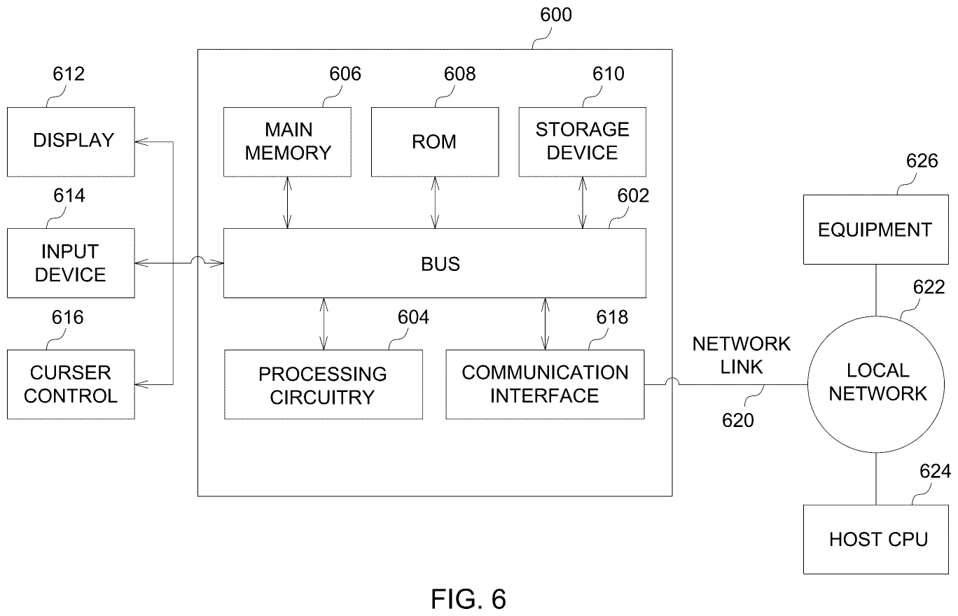

FIG. 6 illustrates a block diagram of a control system with which embodiments may be implemented.

In some embodiments, a control system 600 shown in FIG. 6 may be used to implement the controller 18. The control system 600 may also be an example of any control system described herein.

The control system 600 includes a bus 602 or other communication mechanism for communicating information, and processing circuitry 604 (e.g., at least one processor and/or ASIC) coupled with the bus 602 for processing information. In examples where the processing circuitry 604 is hardware configured to executed stored instructions (e.g., a processor), the control system 600 also includes a main memory 606, such as a random-access memory (RAM) or other dynamic storage device, coupled to the bus 602 for storing information and instructions to be executed by the processing circuitry 604. The main memory 606 also may be used for storing temporary variables or other intermediate information during execution of instructions to be executed by the processing circuitry 604. The control system 600 further includes a read only memory (ROM) 608 or other static storage device coupled to the bus 602 for storing static information and instructions for the processing circuitry 604. A data storage device 610, such as a magnetic disk or optical disk, may be provided and coupled to the bus 602 for storing information and instructions.

The control system 600 may be coupled via the bus 602 to a display 612, such as a flat panel, for displaying information to a user. An input/output device 614, such as a touchscreen, is coupled to the bus 602 for communicating information and command selections to processing circuitry 604. Another type of user input device is cursor control 616, such as a mouse, a trackball, or cursor direction keys for communicating direction information and command selections to processing circuitry 604 and for controlling cursor movement on display 612.

While the display 612 and I/O device 614 are shown outside of the control system 600, it should be understood that the display 612 and the I/O device 614 are part of the control system 600. Moreover, while the display 612, the I/O device 614 and the cursor control 616 are illustrated as separate components, it should be understood that they may be combined, such as a touch screen display.

In some embodiments, the control system 600 can be used to perform various functions described herein. According to some embodiments, such use is provided by control system 600 in response to the processing circuitry 604 executing one or more sequences of one or more instructions contained in the main memory 606. Those skilled in the art will know how to prepare such instructions based on the functions, algorithms and methods described herein. Such instructions may be read into the main memory 606 from another processor-readable medium, such as storage device 610. Execution of the sequences of instructions contained in the main memory 606 causes the processing circuitry 604 to perform the process steps described herein. One or more processors in a multi-processing arrangement may also be employed to execute the sequences of instructions contained in the main memory 606. In alternative embodiments, hard-wired circuitry may be used in place of or in combination with software instructions to implement the various embodiments described herein. Thus, embodiments are not limited to any specific combination of hardware circuitry and software.

Transmission media includes coaxial cables, copper wire and fiber optics, including the wires that comprise the bus 602. Transmission media can also take the form of acoustic or light waves, such as those generated during radio wave and infrared data communications.

Various forms of processor-readable media may be involved in carrying one or more sequences of one or more instructions to the processing circuitry 604 for execution. For example, the instructions may initially be carried on a magnetic disk of a remote computer. The remote computer can load the instructions into its dynamic memory and send the instructions over a network, such as the Internet or a local network. A receiving unit local to the control system 600 can receive the data from the network and provide the data on the bus 602. The bus 602 carries the data to the main memory 606, from which the processing circuitry 604 retrieves and executes the instructions. The instructions received by the main memory 606 may optionally be stored on the storage device 610 either before or after execution by the processing circuitry 604.

The control system 600 also includes a communication interface 618 coupled to the bus 602. The communication interface 618 provides a two-way data communication coupling to a network link 620 that is connected to a local network 622. For example, the communication interface 618 may be an integrated services digital network (ISDN) card or a modem to provide a data communication connection to a corresponding type of telephone line. As another example, the communication interface 618 may be a local area network (LAN) card to provide a data communication connection to a compatible LAN. Wireless links may also be implemented. In any such implementation, the communication interface 618 sends and receives electrical, electromagnetic or optical signals that carry data streams representing various types of information.

The network link 620 typically provides data communication through one or more networks to other devices. For example, the network link 620 may provide a connection through local network 622 to a host computer 624 or to equipment 626 such as a radiation beam source or a switch operatively coupled to a radiation beam source. The data streams transported over the network link 620 can comprise electrical, electromagnetic or optical signals. The signals through the various networks and the signals on the network link 620 and through the communication interface 618, which carry data to and from the control system 600, are exemplary forms of carrier waves transporting the information. The control system 600 can send messages and receive data, including program code, through the network(s), the network link 620, and the communication interface 618.

Although the terms first, second, etc. may be used herein to describe various elements, these elements should not be limited by these terms. These terms are only used to distinguish one element from another. For example, a first element could be termed a second element, and similarly, a second element could be termed a first element, without departing from the scope of this disclosure. As used herein, the term “and/or,” includes any and all combinations of one or more of the associated listed items.

When an element is referred to as being “connected,” or “coupled,” to another element, it can be directly connected or coupled to the other element or intervening elements may be present. By contrast, when an element is referred to as being “directly connected,” or “directly coupled,” to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting. As used herein, the singular forms “a,” “an,” and “the,” are intended to include the plural forms as well, unless the context clearly indicates otherwise. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Specific details are provided in the following description to provide a thorough understanding of example embodiments. However, it will be understood by one of ordinary skill in the art that example embodiments may be practiced without these specific details. For example, systems may be shown in block diagrams so as not to obscure the example embodiments in unnecessary detail. In other instances, well-known processes, structures and techniques may be shown without unnecessary detail in order to avoid obscuring example embodiments.

As discussed herein, illustrative embodiments are described with reference to acts and symbolic representations of operations (e.g., in the form of flow charts, flow diagrams, data flow diagrams, structure diagrams, block diagrams, etc.) that may be implemented as program modules or functional processes include routines, programs, objects, components, data structures, etc., that perform particular tasks or implement particular abstract data types and may be implemented using existing hardware, for example, processing or control circuitry such as, but not limited to, one or more processors, one or more Central Processing Units (CPUs), one or more controllers, one or more arithmetic logic units (ALUs), one or more digital signal processors (DSPs), one or more microcomputers, one or more field programmable gate arrays (FPGAs), one or more System-on-Chips (SoCs), one or more programmable logic units (PLUS), one or more microprocessors, one or more Application Specific Integrated Circuits (ASICs), or any other device or devices capable of responding to and executing instructions in a defined manner.

Although a flow chart may describe the operations as a sequential process, many of the operations may be performed in parallel, concurrently or simultaneously. In addition, the order of the operations may be re-arranged. A process may be terminated when its operations are completed but may also have additional steps not included in the figure. A process may correspond to a method, function, procedure, subroutine, subprogram, etc. When a process corresponds to a function, its termination may correspond to a return of the function to the calling function or the main function.

As disclosed herein, the term “memory,” “storage medium,” “processor readable medium,” “computer readable storage medium” or “non-transitory computer readable storage medium” may represent one or more devices for storing data, including read only memory (ROM), random access memory (RAM), magnetic RAM, core memory, magnetic disk storage mediums, optical storage mediums, flash memory devices and/or other tangible machine-readable mediums for storing information. The term “computer-readable medium” may include, but is not limited to, portable or fixed storage devices, optical storage devices, and various other mediums capable of storing, containing or carrying instruction(s) and/or data.

Furthermore, example embodiments may be implemented by hardware, software, firmware, middleware, microcode, hardware description languages, or any combination thereof. When implemented in software, firmware, middleware or microcode, the program code or code segments to perform the necessary tasks may be stored in a machine or computer readable medium such as a computer readable storage medium. When implemented in software, a processor or processors will perform the necessary tasks. For example, as mentioned above, according to one or more example embodiments, at least one memory may include or store computer program code, and the at least one memory and the computer program code may be configured to, with at least one processor, cause a network element or network device to perform the necessary tasks. Additionally, the processor, memory and example algorithms, encoded as computer program code, serve as means for providing or causing performance of operations discussed herein.

The terms “including” and/or “having,” as used herein, are defined as comprising (i.e., open language). The term “coupled,” as used herein, is defined as connected, although not necessarily directly, and not necessarily mechanically. Terminology derived from the word “indicating” (e.g., “indicates” and “indication”) is intended to encompass all the various techniques available for communicating or referencing the object/information being indicated. Some, but not all, examples of techniques available for communicating or referencing the object/information being indicated include the conveyance of the object/information being indicated, the conveyance of an identifier of the object/information being indicated, the conveyance of information used to generate the object/information being indicated, the conveyance of some part or portion of the object/information being indicated, the conveyance of some derivation of the object/information being indicated, and the conveyance of some symbol representing the object/information being indicated.

Benefits, other advantages, and solutions to problems have been described above with regard to specific embodiments. However, the benefits, advantages, solutions to problems, and any element(s) that may cause or result in such benefits, advantages, or solutions, or cause such benefits, advantages, or solutions to become more pronounced are not to be construed as a critical, required, or essential feature or element of any or all the claims.

### NON-LIMITING ILLUSTRATIVE EMBODIMENTS

The following is a list of non-limiting illustrative embodiments disclosed herein:

Illustrative embodiment 1 includes a radiotherapy system including a radiation system configured to generate a plurality of radiation beams and emit radiation to a patient, the emitted radiation including the plurality of radiation beams; a detector configured to detect at least a portion of emitted radiation that passes through the patient; and a controller configured to detect movement of the patient based on the detected radiation and control movement of at least a portion of the radiation system based on the detected movement of the patient.

Illustrative embodiment 2 includes the radiation system of illustrative embodiment 1, wherein the radiation system is configured to generate the plurality of radiation beams to be parallel and emit the plurality of radiation beams in parallel to the patient.

Illustrative embodiment 3 includes the radiation system of any one of illustrative embodiments 1 and 2, wherein the controller is configured to control the radiation system such that the plurality of radiation beams deliver a radiation rate of less than 40 grays per second (Gy/s).

Illustrative embodiment 4 includes the radiation system of any one of illustrative embodiments 1-3, wherein the controller is configured to control the radiation system such that the plurality of radiation beams emit the radiation to the patient for greater than 2 seconds.

Illustrative embodiment 5 includes the radiation system of any one of illustrative embodiments 1-4, wherein the controller is configured to reconstruct a first image based on detected radiation at a first instance and reconstruct a second image based on detected radiation at a second instance, determine a difference between the first image and the second image, and control the movement of the portion of the radiation system based on the determined difference.

Illustrative embodiment 6 includes the radiation system of any one of illustrative embodiments 1-5, wherein the detected radiation is separate from the plurality of radiation beams.

Illustrative embodiment 7 includes the radiation system of any one of illustrative embodiments 1-6, the radiation system includes a radiation source configured to emit a first radiation beam onto a target to form a focal spot on the target, the target configured to convert the first radiation beam into a radiation field, the focal spot being a source of the radiation field; and a collimator configured to convert the radiation field into at least the plurality of radiation beams.

Illustrative embodiment 8 includes the radiation system of illustrative embodiment 7, wherein the radiation system further includes a deflector configured to alter the first radiation beam, wherein the controller is configured to control the deflector based on the detected radiation to change a location of the focal spot.

Illustrative embodiment 9 includes the radiation system of illustrative embodiment 8, wherein the controller is configured to control the radiation system such that the plurality of radiation beams deliver a reduced radiation rate when the location of the focal spot is changing.

Illustrative embodiment 10 includes the radiation system of illustrative embodiment 7, wherein the target is a cylinder having a diameter less than 50 cm.

Illustrative embodiment 11 includes a method of controlling movement of at least a portion of a radiotherapy system, the method including generating a plurality of radiation beams and emitting radiation to a patient, the emitted radiation including the plurality of radiation beams; detecting at least a portion of emitted radiation that passes through the patient; and controlling movement of the portion of the radiotherapy system based on a detected movement of the patient, the controlling the movement of the portion of the radiotherapy system including detecting the movement of the patient based on the detected radiation.

Illustrative embodiment 12 includes the method of illustrative embodiment 11, wherein the generating generates the plurality of radiation beams to be parallel and the emitting emits the plurality of radiation beams in parallel to the patient.

Illustrative embodiment 13 includes the method of any of illustrative embodiments 11-12, wherein the controlling controls the radiotherapy system such that the plurality of radiation beams deliver a radiation rate of less than 40 grays per second (Gy/s).

Illustrative embodiment 14 includes the method of any of illustrative embodiments 11-13, wherein the controlling controls the radiotherapy system such that the plurality of radiation beams emit the radiation to the patient for greater than 2 seconds.

Illustrative embodiment 15 includes the method of illustrative embodiment 11, wherein the controlling includes reconstructing a first image based on detected radiation at a first instance and reconstructing a second image based on detected radiation at a second instance, determining a difference between the first image and the second image, and controlling the movement of the portion of the radiotherapy system based on the determined difference.

Illustrative embodiment 16 includes the method of any of illustrative embodiments 11-15, wherein the detected radiation is separate from the plurality of radiation beams.

Illustrative embodiment 17 includes the method of any of illustrative embodiments 11-16, wherein the generating includes emitting a first radiation beam onto a target to form a focal spot on the target, converting the first radiation beam into a radiation field, the focal spot being a source of the radiation field, and converting the radiation field into at least the plurality of radiation beams.

Illustrative embodiment 18 includes the method of illustrative embodiment 17, wherein the controlling includes altering the first radiation beam using a deflector, and controlling the deflector based on the detected radiation to change a location of the focal spot.

Illustrative embodiment 19 includes the method of illustrative embodiment 18, wherein the controlling controls the radiotherapy system such that the plurality of radiation beams deliver a reduced radiation rate during the altering.

Illustrative embodiment 20 includes the method of illustrative embodiment 17, wherein the target is a cylinder having a diameter less than 50 cm.

What is claimed is:

1. A radiotherapy system comprising:
a radiation system configured to generate a plurality of radiation beams and emit radiation to a patient, the emitted radiation including the plurality of radiation beams;
a detector configured to detect at least a portion of emitted radiation that passes through the patient; and
a controller configured to detect movement of the patient based on the detected radiation and control movement of at least a portion of the radiation system based on the detected movement of the patient.

2. The radiotherapy system of claim 1, wherein the radiation system is configured to generate the plurality of radiation beams to be parallel and emit the plurality of radiation beams in parallel to the patient.

3. The radiotherapy system of claim 2, wherein the controller is configured to control the radiation system such that the plurality of radiation beams deliver a radiation rate of less than 40 grays per second (Gy/s).

4. The radiotherapy system of claim 3, wherein the controller is configured to control the radiation system such that the plurality of radiation beams emit the radiation to the patient for greater than 2 seconds.

5. The radiotherapy system of claim 1, wherein the controller is configured to,
reconstruct a first image based on detected radiation at a first instance and reconstruct a second image based on detected radiation at a second instance,
determine a difference between the first image and the second image, and
control the movement of the portion of the radiation system based on the determined difference.

6. The radiotherapy system of claim 1, wherein the detected radiation is separate from the plurality of radiation beams.

7. The radiotherapy system of claim 1, wherein the radiation system includes,
a radiation source configured to emit a first radiation beam onto a target to form a focal spot on the target,
the target configured to convert the first radiation beam into a radiation field, the focal spot being a source of the radiation field; and
a collimator configured to convert the radiation field into at least the plurality of radiation beams.

8. The radiotherapy system of claim 7, wherein the radiation system further includes,
a deflector configured to alter the first radiation beam, wherein the controller is configured to control the deflector based on the detected radiation to change a location of the focal spot.

9. The radiotherapy system of claim 8, wherein the controller is configured to control the radiation system such that the plurality of radiation beams deliver a reduced radiation rate when the location of the focal spot is changing.

10. The radiotherapy system of claim 7, wherein the target is a cylinder having a diameter less than 50 cm.

11. A method of controlling movement of at least a portion of a radiotherapy system, the method comprising:
generating a plurality of radiation beams and emitting radiation to a patient, the emitted radiation including the plurality of radiation beams;
detecting at least a portion of emitted radiation that passes through the patient; and
controlling movement of the portion of the radiotherapy system based on a detected movement of the patient, the controlling the movement of the portion of the radiotherapy system including detecting the movement of the patient based on the detected radiation.

12. The method of claim 11, wherein the generating generates the plurality of radiation beams to be parallel and the emitting emits the plurality of radiation beams in parallel to the patient.

13. The method of claim 12, wherein the controlling controls the radiotherapy system such that the plurality of radiation beams deliver a radiation rate of less than 40 grays per second (Gy/s).

14. The method of claim 13, wherein the controlling controls the radiotherapy system such that the plurality of radiation beams emit the radiation to the patient for greater than 2 seconds.

15. The method of claim 11, wherein the controlling includes,
reconstructing a first image based on detected radiation at a first instance and reconstructing a second image based on detected radiation at a second instance,
determining a difference between the first image and the second image, and
controlling the movement of the portion of the radiotherapy system based on the determined difference.

16. The method of claim 11, wherein the detected radiation is separate from the plurality of radiation beams.

17. The method of claim 11, wherein the generating includes,
emitting a first radiation beam onto a target to form a focal spot on the target,
converting the first radiation beam into a radiation field, the focal spot being a source of the radiation field, and
converting the radiation field into at least the plurality of radiation beams.

18. The method of claim 17, wherein the controlling includes,
altering the first radiation beam using a deflector, and
controlling the deflector based on the detected radiation to change a location of the focal spot.

19. The method of claim 18, wherein the controlling controls the radiotherapy system such that the plurality of radiation beams deliver a reduced radiation rate during the altering.

20. The method of claim 17, wherein the target is a cylinder having a diameter less than 50 cm.
